(19) United States
(12) Patent Application Publication
(10) Pub. No.: US 2008/0131497 A1
(43) Pub. Date: Jun. 5, 2008

(54) Formulations of DNase and Methods of Use Thereof

(76) Inventors: Walter R. Perkins, Pennington, NJ (US); Vladimir Malinin, Plainsboro, NJ (US); Paul R. Meers, Princeton Junction, NJ (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/864,127

(22) Filed: Sep. 28, 2007

Related U.S. Application Data
(60) Provisional application No. 60/847,833, filed on Sep. 28, 2006.
(60) Provisional application No. 60/853,265, filed on Oct. 20, 2006.

(51) Int. Cl. A61K 9/127; A61K 38/54; A61K 9/12; A61P 11/00; C12N 9/96

(52) U.S. Cl. 424/450; 424/94.3; 424/45; 435/188

(57) ABSTRACT

Compositions comprising DNase encapsulated in a liposome are provided. Additionally, provided are compositions comprising a free enzyme and empty liposomes, compositions comprising a free enzyme and an antiinfective encapsulated in a liposome, compositions comprising a free enzyme, a free-antiinfective, and empty liposomes, compositions comprising a free enzyme, a free antiinfective, and an antiinfective encapsulated in a liposome, and a composition comprising a free enzyme, empty liposomes, and an antiinfective encapsulated in a liposome, and compositions comprising a free enzyme, a free antiinfective, empty liposomes, an antiinfective encapsulated in a liposome, and pharmaceutical compositions comprising the aforementioned compositions. The Also provided are methods of treating pneumonia, bronchitis, cystic fibrosis, or emphysema comprising administering to a subject in need thereof a therapeutically effective amount of the aforementioned pharmaceutical composition.

### RELATED APPLICATIONS

This application claims the benefit of priority of U.S. Provisional Application Ser. No. 60/847,833, filed on Sep. 28, 2006, and U.S. Provisional Application Ser. No. 60/853,265, filed on Oct. 20, 2006.

### BACKGROUND OF THE INVENTION

DNase is a phosphodiesterase capable of hydrolyzing polydeoxyribonucleic acid. DNase has been purified from various species to various degrees. The complete amino acid sequence for a mammalian DNase was first made available in 1973. See e.g., Liao, et al., J. Biol. Chem. 248:1489 (1973).

DNase has a number of known utilities and has been used for therapeutic purposes. Its principal therapeutic use has been to reduce the viscoelasticity of pulmonary secretions in such diseases as pneumonia and cystic fibrosis, thereby aiding in the clearing of respiratory airways. See e.g., Lourenco, et al., Arch. Intern. Med. 142:2299 (1982); Shak, et al., Proc. Nat. Acad. Sci. 87:9188 (1990); Hubbard, et al., New Engl. J. Med. 326:812 (1992).

DNA encoding human DNase I has been isolated and sequenced and that DNA has been expressed in recombinant host cells, thereby enabling the production of human DNase in commercially useful quantities. See e.g., Shak, et al., Proc. Nat. Acad. Sci. 87:9188-9192 (1990). Recombinant human DNase (rhDNase) has been found to be useful clinically, especially in purified form such that the DNase is free from proteases and other proteins with which it is ordinarily associated in nature. See e.g., Hubbard, et al., New Engl. J. Med. 326:812 (1992).

The means and methods by which human DNase can be obtained in pharmaceutically effective form is described in the patent applications cited above. Various specific methods for the purification of DNase are known in the art. See e.g., Khouw, et al., U.S. Pat. No. 4,065,355 (issued Dec. 27, 1977); Markey, FEBS Letters 167:155 (1984); Nefsky, et al., Eur. J. Biochem. 179:215 (1989).

Although it was not appreciated at the time the above-referenced patent applications were filed, the DNase product obtained from cultures of recombinant host cells typically comprises a mixture of deamidated and non-deamidated forms of DNase. The existence of deamidated forms of DNase remained unappreciated notwithstanding that the phenomenon of deamidation of asparagine and glutamine residues in some proteins is known. See e.g., Eipper et al., Ann. Rev. Physiol. 50:333 (1988); Kossiakoff, Science 240:191 (1988); Bradbury et al., Trends in Biochem. Sci. 16:112 (1991); and Wright, Protein Engineering 4:283 (1991).

Cystic fibrosis (CF), also called mucoviscidosis, is an autosomal, recessive, hereditary disease of the exocrine glands. It affects the lungs, sweat glands and the digestive system, causing chronic respiratory and digestive problems. It is caused by mutations in the cystic fibrosis transmembrane conductance regulator (CFTR) protein. It is the most common fatal autosomal recessive diseases amongst Caucasians.

The first manifestation of CF is sometimes meconiumileus, occurring in 16% of infants who develop CF. Other symptoms of CF manifest during early childhood. Both lungs and pancreas produce abnormally viscous mucus. This mucus begins to build up and starts to clog the opening to the pancreas and the lungs. Pulmonary problems start from the constant presence of thick, sticky mucus and are one of the most serious complications of CF. The standard for measuring pulmonary function is forced expiratory volume in one second (FEV1). FEV1 is often expressed as a percent of that predicted for a healthy individual based on height, weight, gender and age. Cystic fibrosis patients often have low FEV1 values compared to their healthier counterparts

Daily aerosol breathing treatments are very commonly prescribed for CF patients. Aerosolized medicines commonly given include albuterol, ipratropium bromide and Pulmozyme to loosen secretions and decrease inflammation. Lytic agent Pulmozyme (recombinant human Dnase) administered by inhalation shows an improvement in FEV1 of approximately 10%. Pulmozyme acts by breaking up the fibrous DNAs in the respiratory mucoid system. Another lytic agent, alginase has shown to enhance phagocytosis of Pseudomonas aerugionosa by human monocyte-derived macrophage. Eftekhar, F., Speert, D. P. Infect. Immun., 1985, 56 (11), 2788. Antibiotics such as tobramycin adminstered by inhalation has also improved FEV1 by approximately 10%, thought mainly to be due to the substantial reduction in colony forming units (CFU) of about 2 log units.

CF patients are typically hospitalized somewhat regularly, often every 6 months depending on the severity of the case. Additionally, other pulmonary diseases of similar symptoms exist such as pneumonia, bronchitis, or emphysema. New methods for increasing pulmonary functions of patients suffering from pulmonary distress are needed.

### SUMMARY OF THE INVENTION

It is an object to provide a composition that provides effective delivery of enzymes such as lytic agents such as DNase to sites of pulmonary distress.

It is also an object to provide a method of treating pneumonia, bronchitis, cystic fibrosis, or emphysema comprising administering to a patient in need thereof a therapeutically effective amount of the aforementioned composition.

It is also an object to provide compositions that combine multiple agents that act synergistically in clearing a subject's airway.

It is also an object to provide compositions that provide effective delivery of an agent, or agents, to sites of pulmonary distress.

It is also an object to provide a method of stabilizing enzyme activity over time, and to stabilize enzyme activity during and after nebulization.

In one aspect, the disclosure relates to a composition comprising DNase encapsulated in a liposome.

In a further embodiment, the liposome comprises lipids selected from the group consisting of phospholipids, tocopherols, sterols, glycoproteins, and mixtures thereof.

In a further embodiment, the liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof.

In a further embodiment, the liposome comprises phospholipids and a sterol. In a further embodiment, the liposome comprises dioleylphosphatidylcholine (DOPC), dioleylphosphatidylglycerol (DOPG), and cholesterol. In a further embodiment, the ratio of DOPC:DOPG:cholesterol is 60-70:1-10:25-35 by mole percent. In a further embodiment, the ratio of DOPC:DOPG:cholesterol is 65:5:30 by mole percent. In a further embodiment, the liposome comprises lipids in a 20-30:1 ratio by weight with DNase. In a further embodiment, the liposome comprises lipids in a 25:1 ratio by weight with DNase.

In a further embodiment, the liposome comprises DOPC, DOPG, and cholesterol at 65:5:30 mole percent, and wherein the weight ratio of DOPC, DOPG, and cholesterol to DNase is 25:1.

In a further embodiment, the aforementioned compositions further comprise a free enzyme, free antiinfective, empty liposomes, or antiinfective encapsulated in a second liposome.

In a further embodiment, the aforementioned compositions further comprise a free enzyme and a free antiinfective, or a free enzyme and empty liposomes, or a free enzyme and an antiinfective encapsulated in a second liposome.

In a further embodiment, the aforementioned compositions further comprise a free antiinfective and empty liposomes, or a free antiinfective and an antiinfective encapsulated in a second liposome.

In a further embodiment, the aforementioned compositions further comprise empty liposomes and an antiinfective encapsulated in a second liposome.

In a further embodiment, the aforementioned compositions further comprise a free enzyme, a free antiinfective, and empty liposomes.

In a further embodiment, the aforementioned compositions further comprise a free enzyme, a free antiinfective, and an antiinfective encapsulated in a second liposome.

In a further embodiment, the aforementioned compositions further comprise a free antiinfective, empty liposomes, and an antiinfective encapsulated in a second liposome.

In a further embodiment, the aforementioned compositions further comprise a free enzyme, a free antiinfective, empty liposomes, and an antiinfective encapsulated in a second liposome.

In the preceeding embodiments, the free enzyme may be a lytic agent such as, for example, DNase. The antiinfective, free or encapsulated, may be an aminoglycoside, such as, for example, amikacin, gentamicin, or tobramycin. In one embodiment, the antiinfectives are amikacin.

In the preceeding embodiments, the empty liposomes or the second liposome may comprise lipids selected from the group consisting of phospholipids, tocopherols, sterols, glycoproteins, and mixtures thereof. In a further embodiment, the empty liposomes or second liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof. In a further embodiment, the empty liposomes or second liposome comprises phospholipids and sterol.

For example, in one embodiment the aforementioned liposomal DNase compositions further comprise free DNase.

In another embodiment the aforementioned liposomal DNase compositions further comprise free amikacin.

In another embodiment the aforementioned liposomal DNase compositions further comprise empty liposomes comprising phospholipids and sterol.

In another embodiment the aforementioned liposomal DNase compositions further comprise amikacin encapsulated in a liposome comprising phospholipids and sterol.

In another embodiment the aforementioned liposomal DNase compositions further comprise free DNase and free amikacin.

In another embodiment the aforementioned liposomal DNase compositions further comprise free DNase and empty liposomes comprising phospholipids and sterol.

In another embodiment the aforementioned liposomal DNase compositions further comprise free DNase and amikacin encapsulated in a liposome comprising phospholipids and sterol.

In another embodiment the aforementioned liposomal DNase compositions further comprise free amikacin and empty liposomes comprising phospholipids and sterol.

In another embodiment the aforementioned liposomal DNase compositions further comprise free amikacin and amikacin encapsulated in a liposome comprising phospholipids and sterol.

In another embodiment the aforementioned liposomal DNase compositions further comprise empty liposomes comprising phospholipids and sterol and amikacin encapsulated in a liposome comprising phospholipids and sterol.

In another embodiment the aforementioned liposomal DNase compositions further comprise free DNase, free amikacin, and empty liposomes comprising phospholipids and sterol.

In another embodiment the aforementioned liposomal DNase compositions further comprise free DNase, free amikacin, and amikacin encapsulated in a liposome comprising phospholipids and sterol.

In another embodiment the aforementioned liposomal DNase compositions further comprise free amikacin, empty liposomes comprising phospholipids and sterol, and amikacin encapsulated in a liposome comprising phospholipids and sterol.

In another embodiment the aforementioned liposomal DNase compositions further comprise free DNase, free amikacin, empty liposomes comprising phospholipids and sterol, and amikacin encapsulated in a liposome comprising phospholipids and sterol.

In another aspect, the disclosure relates to a composition comprising an enzyme and empty liposomes.

In another aspect, the disclosure relates to a composition comprising an enzyme and an antiinfective encapsulated in a liposome.

In another aspect, the disclosure relates to a composition comprising an enzyme, a free antiinfective, and empty liposomes.

In another aspect, the disclosure relates to a composition comprising an enzyme, a free antiinfective, and an antiinfective encapsulated in a liposome.

In another aspect, the disclosure relates to a composition comprising an enzyme, empty liposomes, and an antiinfective encapsulated in a liposome.

In another aspect, the disclosure relates to a composition comprising an enzyme, free antiinfective, empty liposomes, and an antiinfective encapsulated in a liposome.

In the preceeding aspects, the enzyme may be a lytic agent, such as, for example, DNase. The antiinfective, free or encapsulated, may be an aminoglycoside, such as, for example, amikacin, gentamicin, or tobramycin. In one embodiment, the antiinfectives are amikacin.

In the preceeding aspects, the empty liposomes or the second liposome may comprise lipids selected from the group consisting of phospholipids, tocopherols, sterols, glycoproteins, and mixtures thereof. In a further embodiment, the empty liposomes or second liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof. In a further embodiment, the empty liposomes or second liposome comprises phospholipids and sterol.

For example, in one embodiment the disclosure relates to a composition comprising DNase and empty liposomes comprising phospholipids and sterol.

In another embodiment the disclosure relates to a composition comprising DNase and amikacin encapsulated in a liposome comprising phospholipids and sterol.

In another embodiment the disclosure relates to a composition comprising DNase, free amikacin, and empty liposomes comprising phospholipids and sterol.

In another embodiment the disclosure relates to a composition comprising DNase, free amikacin, and amikacin encapsulated in a liposome comprising phospholipids and sterol.

In another embodiment the disclosure relates to a composition comprising DNase, empty liposomes comprising phospholipids and sterol, and amikacin encapsulated in a liposome comprising phospholipids and sterol.

In another embodiment the disclosure relates to a composition comprising DNase, free amikacin, empty liposomes comprising phospholipids and sterol, and amikacin encapsulated in a liposome comprising phospholipids and sterol.

In another aspect, the disclosure relates to a pharmaceutical composition comprising any of the aforementioned compositions and a pharmaceutically acceptable carrier.

In another aspect, the disclosure relates to a method of treating pneumonia, bronchitis, cystic fibrosis, or emphysema in a subject comprising administering to a subject in need thereof a therapeutically effective amount of the aforementioned pharmaceutical composition.

In a further embodiment, the pharmaceutical composition is administered by aerolization.

In a further embodiment, the pharmaceutical composition is administered daily.

In a further embodiment, the subject is a primate, bovine, ovine, equine, porcine, rodent, feline, or canine. In a further embodiment, the subject is a human.

In another aspect, the disclosure relates to a method of stabilizing the activity of an enzyme comprising storing the enzyme in the presence of a liposome. In a further embodiment, the enzyme is a lytic agent. In a further embodiment, the enzyme is DNase.

In a further embodiment, the liposome is an empty liposome.

In a further embodiment, the liposome encapsulates an antiinfective. In a further embodiment, the liposome encapsulates amikacin, gentamicin, or tobramycin. In a further embodiment, the liposome encapsulates amikacin.

In a further embodiment, the liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof.

In a further embodiment, the liposome comprises phospholipids and sterol.

In a further embodiment, storing is conducted over 1 or 2 weeks.

In another aspect, the disclosure features a method of stabilizing the activity of an enzyme during and after nebulization comprising nebulizing the enzyme in the presence of a liposome. In a further embodiment, the enzyme is a lytic agent. In a further embodiment, the enzyme is DNase.

In a further embodiment, the liposome is an empty liposome.

In a further embodiment, the liposome encapsulates an antiinfective. In a further embodiment, the liposome encapsulates amikacin, gentamicin, or tobramycin. In a further embodiment, the liposome encapsulates amikacin.

In a further embodiment, the liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof. In a further embodiment, the liposome comprises phospholipids and sterol.

These embodiments of the present invention, other embodiments, and their features and characteristics, will be apparent from the description, drawings and claims that follow.

### BRIEF DESCRIPTION OF THE DRAWINGS

FIG. 1 depicts the chromatogram profile for the preparation of liposomal encapsulated DNase. The first peak represents DNase encapsulated into liposomes while the second peak is free DNase.

FIG. 2 depicts the stabilizing effects empty liposomes and liposomal encapsulated antiinfective have on enzyme activity over time.

FIG. 3 depicts a viscosity v. time graph for a DNase activity assay.

FIG. 4 depicts the stabilizing effects liposomal encapsulated antiinfective has on enzymes during and after nebulization.

### DETAILED DESCRIPTION OF THE INVENTION

### I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “bioactive agent” as used herein refers to small molecules or macromolecules with biological activity such as drugs or prodrugs. Bioactive agent, as used herein, does not include pharmaceutical excipients.

The term “bioavailable” is art-recognized and refers to a form of the subject invention that allows for it, or a portion of the amount administered, to be absorbed by, incorporated to, or otherwise physiologically available to a subject or patient to whom it is administered.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

The term “empty” as used herein to describe liposomes refers to liposomes without encapsulated bioactive agent. An empty liposome may still of course have other things encapsulated along the lines of a pharmaceutical carrier such as, for example, water.

The term “free” as used herein to describe either a lytic agent such as DNase or antiinfective refers to DNase or bioactive agents unencapsulated by liposomes.

The term “human DNase” as used herein refers to a polypeptide having the amino acid sequence of human mature DNase I set forth in U.S. Pat. No. 6,932,965, incorporated herein by reference, as well as amino acid sequence variants thereof (including allelic variants) that are enzymatically active in hydrolyzing DNA. Thus, the term “human DNase” herein denotes a broad definition of those materials encapsulated in a liposome. The term “human DNase” as used herein necessarily embraces native mature human DNase having an asparagine (Asn) residue at amino acid position 74 of the polypeptide. That asparagine has been found herein to be susceptible to deamidation, which deamidation may produce a mixture of deamidated and non-deamidated forms of human DNase. Instead of the Asn residue at amino acid position 74, deamidated DNase has an aspartic acid (Asp) or an iso-aspartate (iso-Asp) residue.

The term “deamidated human DNase” as used herein thus means human DNase that is deamidated at the asparagine residue that occurs at position 74 in the amino acid sequence of native mature human DNase. It has been found that deamidated human DNase may arise during the production of human DNase by recombinant means, and may be found in preparations of human DNase obtained from recombinant host cells. Additionally, deamidated human DNase may arise upon in vitro storage of non-deamidated human DNase.

Although the asparagine residue at amino acid position 7 in the amino acid sequence of native mature human DNase also may be deamidated (in addition to the asparagine residue at amino acid position 74), such doubly deamidated DNase has been found to be enzymatically inactive.

The term “including” is used herein to mean “including but not limited to”. “Including” and “including but not limited to” are used interchangeably.

The term “mammal” is known in the art, and exemplary mammals include humans, primates, bovines, porcines, canines, felines, and rodents (e.g., mice and rats).

The term “mixture” as used herein in reference to preparations of human DNase means the presence of both deamidated and non-deamidated forms of human DNase. It has been found, for example, that in preparations of human DNase obtained from recombinant expression, as much as about 50% to 80% or more of the human DNase is deamidated.

A “patient,” “subject” or “host” to be treated by the subject method may mean either a human or non-human animal.

The term “pharmaceutically-acceptable salts” is art-recognized and refers to the relatively non-toxic, inorganic and organic acid addition salts of compounds, including, for example, those contained in compositions of the present invention.

By the term “pharmaceutical carrier” herein is meant a pharmaceutically acceptable material that is employed together with encapsulated or unencapsulated DNase for the proper and successful administration of the DNase to a patient. Suitable carriers are well known in the art, and are described, for example, in the Physicians Desk Reference, the Merck Index, and Remington's Pharmaceutical Sciences.

The term “prodrug” is art-recognized and is intended to encompass compounds which, under physiological conditions, are converted into the antibacterial agents of the present invention. A common method for making a prodrug is to select moieties which are hydrolyzed under physiological conditions to provide the desired compound. In other embodiments, the prodrug is converted by an enzymatic activity of the host animal or the target bacteria.

The term “purified deamidated human DNase” as used herein means deamidated human DNase that is substantially free of non-deamidated human DNase. In other words, non-deamidated human DNase will comprise less than about 10%, preferably less than about 5%, and most preferably less than about 1% by weight of the total DNase in the purified deamidated human DNase composition.

The term “purified non-deamidated human DNase” as used herein means non-deamidated human DNase that is substantially free of deamidated human DNase. In other words, deamidated human DNase will comprise less than about 25%, preferably less than about 5%, and most preferably less than about 1% by weight of the total DNase in the purified non-deamidated human DNase composition.

The term “single dose” is art-recognized and refers to a period of time during which the lipid formulation is being administered, or to the amount of lipid formulation given in that period of time. A single dose may last several seconds, minutes, or hours.

The term “stabilizing” or “stabilization” as used herein to describe enzyme activity refers to the enzyme maintaining more of its activity under a set of conditions than it does when those conditions are absent. For example, the stabilization of an enzyme in the presence of a liposome over time means that the enzyme maintains more activity than it would if stored over the same amount of time in the absence of the liposome. In certain embodiments, stabilizing or stabilization means that an enzyme maintains 85%, 90%, 95%, 96%, 97%, 98%, or 99% or more of its activity.

The term “treating” is art-recognized and refers to curing as well as ameliorating at least one symptom of any condition or disorder.

### II. DNase

DNase is recombinant human deoxyribonuclease I, an enzyme which selectively cleaves DNA. DNase hydrolyzes the DNA present in sputum/mucus of cystic fibrosis patients and reduces viscoelasticity in the lungs, promoting improved clearance of secretions. DNase is produced in Chinese hamster ovary cells.

DNase is the most recent therapeutic agent developed with this basic mechanism of action. Prior to the cloning of the human enzyme, bovine DNase was on the market for many years, though its utility was limited by the inherent antigenic response to a cow protein in the lungs of patients.

After the successful cloning and expression of human DNase in recombinant host cells, it was discovered that the DNase product obtained from such recombinant expression typically existed as a mixture of as then yet undefined components. In particular, isoelectric focusing (IEF) analysis of human DNase purified from cultures of recombinant Chinese hamster ovary (CHO) cells revealed a complex pattern of DNase species. The various DNase species were determined to result from several post-translational modifications of the DNase, including deamidation.

Two assays were used to determine the presence and extent of deamidated DNase in such preparations. One method involved tryptic digestion of the starting preparation of DNase and analysis of the resulting peptides by reverse phase HPLC. In this method, the amount of deamidated DNase in the starting preparation was determined by measuring the quantities of six deamidation-indicating tryptic peptides.

The other method involved chromatography of the starting preparation of DNase on a tentacle cation exchange (TCX) column. It was discovered that the TCX column is capable of resolving deamidated human DNase and non-deamidated human DNase, such that each form of DNase could be effectively separated from the other, and obtained in purified form. In this method, the amount of deamidated and non-deamidated DNase in the starting preparation was determined by measuring on chromatograms the peak areas corresponding to the separated forms of DNase.

Although these two methods are about equally effective in determining and quantitating deamidated DNase, the TCX method is especially efficient, requiring far less time and labor than the other method. Moreover, TCX chromatography provides a means for separating deamidated and non-deamidated forms of DNase, whereas conventional cation exchange resins and various other chromatography resins that were analyzed were not capable of such separation.

The general principles of TCX chromatography have been described, for example, by Miller, J. Chromatography 510:133 (1990); Janzen et al., J. Chromatography 522:77 (1990); and Hearn et al., J. Chromatography 548:117 (1991). Without limiting the invention to any particular mechanism or theory of operation, it is believed that the Asn-74 residue in human DNase that is susceptible to deamidation is located within the DNA-binding groove of the enzyme, by analogy to the known crystal structure of bovine DNase. The DNA-binding groove contains basic amino acid residues (in order to bind DNA) and this groove apparently is accessible to the ligands of the tentacle cation exchange resin but not to the much shorter ligands of conventional cation exchange resins. Presumably the ligands of the tentacle cation exchange resin mimic natural nucleic acid substrates. Therefore, it is expected that tentacle action exchange chromatography will be useful for the purification of other nucleases, such as ribonuclease (RNase) or restriction endonucleases, as well as DNA binding proteins.

Alternatively, the separation of deamidated and non-deamidated forms of DNase may be accomplished by chromatography using a resin or other support matrix containing covalently bound cationic polymers such as heparin or a synthetic non-hydrolyzable DNA analog.

Immobilized heparin chromatography columns are commercially available (for example, from Toso Haas Co., Montgomeryville, Pa.). Non-hydrolyzable DNA analogs have been described, for example, by Spitzer et al., Nuc. Acid. Res. 16:11691 (1988). An immobilized non-hydrolyzable DNA analog column is conveniently prepared by synthesizing such a DNA analog with an amino acid group at the 3′-end of one or both of its complementary strands. The amino group is then available for coupling to an epoxy-activated column, as described, for example, in literature published by Rainin Biochemical LC Products (Woburn, Mass.).

Following the successful separation of deamidated and non-deamidated human DNases according to the methods of the present invention, it was found that deamidated human DNase has diminished enzymatic activity as compared to non-deamidated human DNase, as determined by a methyl green (MG) assay. Kurnick, Arch. Biochem. 29:41 (1950). It was found that deamidated human DNase exhibits just over half of the enzymatic activity of non-deamidated human DNase. Thus, by combining the purified deamidated DNases and the purified non-deamidated DNase of the present invention in various proportions, it is possible to prepare pharmaceutical compositions of human DNase having any desired specific activity in the range between the specific activities of the individual components, as may be optimal for treating particular disorders.

Pulmozyme® (from Genentech) is a highly purified, commercially available solution of DNase.

### III. Lipids

The lipids used to encapsulate DNase can be synthetic, semi-synthetic or naturally-occurring lipids, including phospholipids, tocopherols, sterols, fatty acids, glycoproteins such as albumin, negatively-charged lipids and cationic lipids. In terms of phosholipids, they could include such lipids as phosphatidylcholine (PC), phosphatidylglycerol (PG), phosphatidylinositol (PI), phosphatidylserine (PS), phosphatidylethanolamine (PE), and phosphatidic acid (PA). The egg counterparts, egg phosphatidylcholine (EPC), egg phosphatidylglycerol (EPG), egg phosphatidylinositol (EPI), egg phosphatidylserine (EPS), egg phosphatidylethanolamine (EPE), and egg phosphatidic acid (EPA); the soya counterparts, soy phosphatidylcholine (SPC), SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphophatidylcholine (DPPC), a major constituent of naturally-occurring lung surfactant. Other examples include dioleylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (Pls) and the phosphatidyl serines (PSs). Examples include DMPG, DPPG, DSPG, DMPA, DPPA, DSPA, DMPI, DPPI, DSPI, DMPS, DPPS and DSPS.

### IV. Liposomes

Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the 60 mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

The liposomes are comprised of particles with a mean diameter of approximately 0.01 microns to approximately 3.0 microns, preferably in the range about 0.1 to 1.0 microns, and even more preferably in the range of about 0.1 to 0.5 microns.

In one embodiment the compositions and methods of present invention comprise a combination of both DNase encapsulated liposomes and so called “empty” liposomes, i.e. liposomes that are not encapsulating bioactive agents but are themselves the bioactive agent. It has recently been postulated that empty liposomes are effective for increasing the forced expiratory volume in one second (FEV1) which is a standard for pulmonary function. FEV1 units are liters and milliliters, and is often expressed as a % of that predicted for healthy individuals based on height, weight, gender, and age. Example 1 discloses the preparation of liposome encapsulated DNase, and Example 2 discloses an experiment supporting the surprising notion that empty liposomes increase FEV1.

### V. Antiinfectives

Antiinfectives are agents that act against infections, such as bacterial, mycobacterial, fungal, viral or protozoal infections. Antiinfectives covered by the invention include but are not limited to aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), sulfonamides (e.g., sulfanilamide, sulfadiazine, sulfamethaoxazole, sulfisoxazole, sulfacetamide, and the like), paraminobenzoic acid, diaminopyrimidines (such as trimethoprim, often used in conjunction with sulfamethoxazole, pyrazinamide, and the like), quinolones (such as nalidixic acid, cinoxacin, ciprofloxacin and norfloxacin and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillin and the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil; cefmetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), betalactamase inhibitors (such as clavulanic acid), chlorampheriicol, macrolides (such as erythromycin, azithromycin, clarithromycin, and the like), lincomycin, clindamycin, spectinomycin, polymyxin B, polymixins (such as polymyxin A, B, C, D, E1 (colistin A), or E2, colistin B or C, and the like) colistin, vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibacterial agent that can be lipid encapsulated. Antiinfectives can include antifungal agents, including polyene antifungals (such as amphotericin B, nystatin, natamycin, and the like), flucytosine, imidazoles (such as n-ticonazole, clotrimazole, econazole, ketoconazole, and the like), triazoles (such as itraconazole, fluconazole, and the like), griseoifulvin, terconazole, butoconazole ciclopirax, ciclopirox olamine, haloprogin, tolnaftate, naftifine, terbinafine, or any other antifungal that can be lipid encapsulated or complexed. Discussion and the examples are directed primarily toward amikacin but the scope of the application is not intended to be limited to this antiinfective. Combinations of drugs can be used.

Particularly preferred antiinfectives include the aminoglycosides, the quinolones, the polyene antifungals and the polymyxins.

Also included as suitable antiinfectives used in the liposomal formulations are pharmaceutically acceptable addition salts and complexes of antiinfectives. In cases wherein the compounds may have one or more chiral centers, unless specified, the present invention comprises each unique racemic compound, as well as each unique nonracemic compound.

In cases in which the antiinfectives have unsaturated carbon-carbon double bonds, both the cis (Z) and trans (E) isomers are within the scope of this invention. In cases wherein the antiinfectives may exist in tautomeric forms, such as keto-enol tautomers, such as

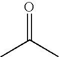

and

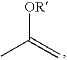

each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included as suitable antiinfectives used in the liposomal formulations are prodrugs of the platinum compounds. Prodrugs are considered to be any covalently bonded carriers which release the active parent compound in vivo.

### VI. Enzyme Stability

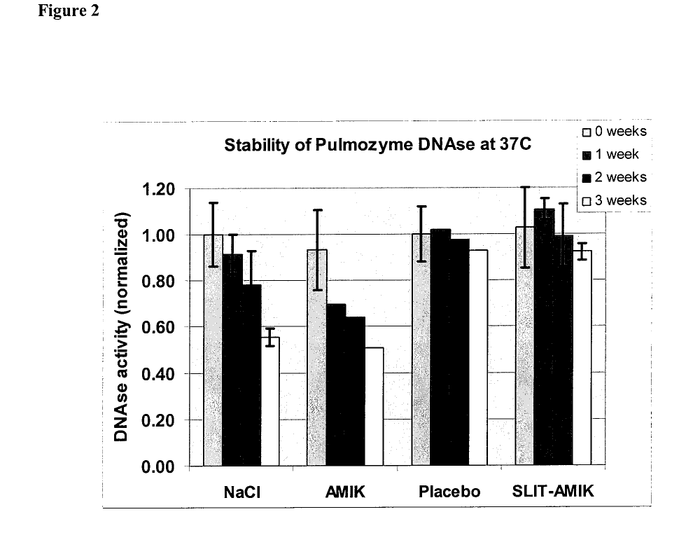

It has now surprisingly been found that enzyme activity is stabilized over time when the enzyme is stored in the presence of a liposomal formulation. Example 3 measures DNase activity over time for 4 solutions. The results are depicted in FIG. 2. DNase activity was measured initially and then after 1 and 2 weeks. The 4 solutions were stored at 37° C. The first solution represented by the first group of 3 bars represents DNase in a 1.5% NaCl solution. The second solution represented by the second group of 3 bars represents DNase in the presence of the antiinfective amikacin at 75 mg/mL. The third solution represented by the third group of 3 bars represents DNase in the presence of empty liposomes (placebo liposomes). The fourth solution represented by the fourth group of 3 bars represents DNase in the presence of liposomal encapsulated amikacin (SLIT-AMIK). For the first and second solutions, DNase activity drops off over the two week period as expected. For the third and fourth solutions where a liposomal formulation is present, there is no reduction in activity as one may expect. The liposomal formulation has imparted stability to the DNase not from encapsulation, but from being present.

It has also suprisingly been found that enzyme activity is stabilized during nebulization when the enzyme is nebulized in the presence of a liposomal formulation. In Example 4, Pulmozyme® was nebulized in an NaCl 1.5% solution alone, and in an NaCl 1.5% solution with a liposomal encapsulated antiinfective. The nebulized aerosol solutions were collected and analyzed for DNase enzymatic activity as described previously.

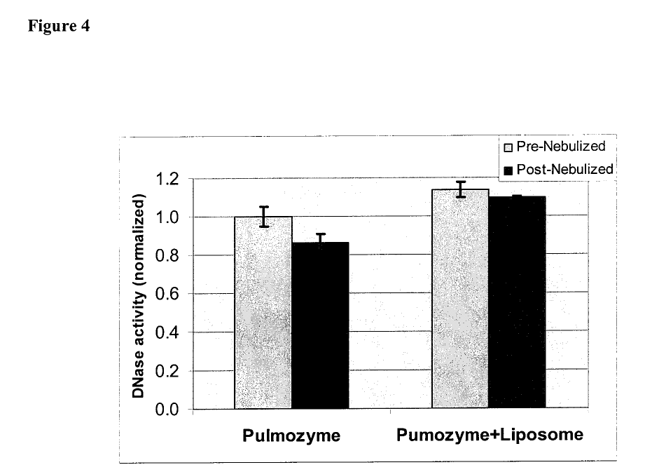

FIG. 4 presents effect of liposomes on DNase stability during nebulization. As one can see, DNase alone loses about 14% activity after nebulization, while DNase mixed with liposomes preserves most its activity. Effect of nebulization on DNase in solution was statistically significant.

Not to be bound by theory, one explanation of the mechanism of protective effect of liposomes may be that nebulization creates large number of small aerosol droplets thus producing a very large area of water-air surface. It is known that enzymes may denature when exposed to water-air surface and lose their enzymatic activity. Liposomes may compete with enzyme molecules for the water-air surface preferentially occupying it and thus preventing access of enzymes to the surface.

### VII. Dosages

The dosage of any formulation of the present invention will vary depending on the symptoms, age and body weight of the patient, the nature and severity of the disorder to be treated or prevented, the route of administration, and the form of the subject composition. Any of the subject compositions may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the composition, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

Generally, the lipid formulation is administered to the subject in need thereof on a daily basis. Daily, the subject may receive at least a single dose of lipid formulation which may last several seconds, minutes, or hours.

### VIII. Pharmaceutical Formulation

The pharmaceutical formulation of the liposomal formulation of the present invention may be comprised of either an aqueous dispersion of the liposomal formulations, or a dehydrated powder containing the liposomal formulations. The formulation may contain lipid excipients to form the liposomes, and salts/buffers to provide the appropriate osmolarity and pH. The dry powder formulations may contain additional excipients to prevent the leakage of encapsulated components during the drying and potential milling steps needed to create a suitable particle size for inhalation (i.e., about 1-5 μm). Such excipients are designed to increase the glass transition temperature of the liposomal formulation. The pharmaceutical excipient may be a liquid or solid filler, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### IX. Inhalation Device

The liposomal formulations of the present invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the liposomal formulations or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydrofluorocarbons (HFCs) such as 134a and 227. Traditional chlorofluorocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

### EXEMPLIFICATION

### Example 1

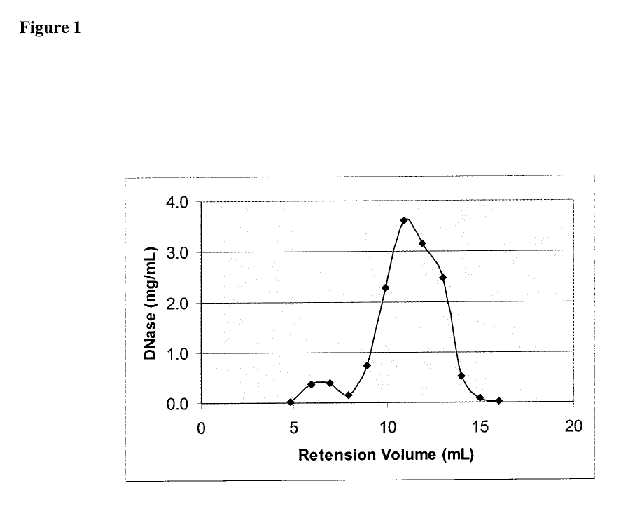

To encapsulate bovine DNase into liposomes extrusion technique was used. Lyophilized lipids 40 mg (POPC/POPG/Chol 65:5:30 mol %) were hydrated with 1 mL solution containing 20 mg bovine DNase, 1 mM CaCl₂ and 0.9% NaCl. After incubating for 1 hour at room temperature, suspension of MLVs was extruded through 0.4 um and then 0.2 um polycarbonate filters using MiniExtruder (Avanti). Unencapsulated DNase was separated by gel filtration with Sephacryl S-500 columnusing 0.9% NaCl as a running phase. Post-column fractions of 1 mL each were collected and analysed for DNase contents. DNase concentration was measured by fluorescence using excitation wavelength 282 nm and emission wave length 332 nm. Chromatogram profile is shown in FIG. 1.

Liposomes collected in fractions 2 and 3 had ˜1 mg DNase (as measured by fluorescence) and ˜25 mg lipids thus making the DNase-to-Lipid ratio about 1:25 by weight.

### Example 2

A study was conducted where several patients with cystic fibrosis were administered lipid formulations comprising 500 mg of DPPC, 250 mg of cholesterol, and 500 mg of entrapped amikacin. The lipid formulation was administered daily for 14 days. The effects of the lipid formulation on FEV1 and colony forming units (CFU) appear in Tables 1 and 2, respectively.

TABLE 1
The effect of the lipid formulation on FEV1.

| | | | FEV1 | | | |
|---|---|---|---|---|---|---|
| | FEV1 | | increase | | | FEV1 |
| | before | FEV1 after | after | FEV1 | | increase |
| | treatment | treatment | treatment | before | FEV1 after | after |
| Patient | (L) | (L) | (mL) | treatment % | treatment % | treatment % |
| A | 1.64 | 1.81 | 170 | 59 | 65 | 6 |
| B | 5.09 | 5.49 | 400 | 114.6 | 123.6 | 9 |
| C | 2.4 | 2.67 | 270 | 76.6 | 85.1 | 8.5 |
| D | 0.88 | 1.22 | 340 | 31 | 43 | 12 |
| | | | | | Ave. | 8.9 |

TABLE 2
The effect of the lipid formulation on CFU.

| | | | log(CFU) | log(CFU) | |
|---|---|---|---|---|---|
| | CFU before | CFU after | before | after | Change in |
| Patient | treatment | treatment | treatment | treatment | log(CFU) |
| A | 2.00E+7 | 4.80E+6 | 7.30 | 6.68 | −0.62 |
| B | 1.00E+5 | 1.00E+6 | 5.00 | 6.00 | +1.00 |
| C | 1.00E+5 | 2.00E+7 | 5.00 | 7.30 | +2.30 |
| D | 2.00E+8 | 2.00E+8 | 8.3 | 8.30 | 0 |
| | | | | Ave. | +0.67 |

The study indicates that the treatment resulted in about a 10% improvement in FEV1 without any meaningful reduction in CFU. It has been reported that treatment with free aminoglycoside by inhalation of CF patients caused a substantial reduction in CFU (approximately 2 log units) as well as approximately a 10% improvement in FEV1. The improved FEV1 values were explained as an overall pulmonary function improvement resulting from the antibiotic activity of the aminoglycoside.

The present study shows that FEV1 improvement was achieved even though there was not enough aminoglycoside to cause noticeable antibiotic activity. This strongly indicates that the lipids are the main cause for FEV1 improvement. Not wanting to be bound by theory, one explanation of this unexpected phenomena is that the liposome associates with the DNA causing condensation of DNA. Another explanation may be that the liposome lubricates the airways.

### Example 3

Stability of enzymes in presence of liposomes. Pulmozyme®(as supplied was mixed with empty liposomes (Placebo, 50 mg/mL lipid concentration) or antiinfective-encapsulated liposomes (Liposome-Amikacin, 50 mg/mL lipid concentration, 75 mg/mL amikacin concentration) at a volume ratio of 2:3 yielding final DNase concentration 0.4 mg/mL and final lipid concentration of ˜30 mg/mL. Liposomes were made as described previously, and had lipid composition DPPC/Cholesterol 2:1 wt/wt. Alternatively, as a control, Pulmozyme® was mixed with NaCl 1.5% solution or amikacin 75 mg/mL solution in NaCl 1.5%. Samples were stored in vials 1 mL each at temperatures 4° C., 25° C. and 37° C.

At specific time points aliquots of samples were taken from the vials, diluted and analyzed for DNase activity. Enzymatic activity of DNase was determined by viscosity assay. DNA decomposition by DNase results in decrease of viscosity. DNase at 1 μg/mL normally can digest 2 mg/mL DNA resulting in decrease of viscosity from ˜25 cP (5 rpm) to 10 cP in ˜10 min (and to approaching 1 cP in longer time) in presence of 1 mM Ca²⁺ and Mg²⁺ ions. Kinetics of viscosity change due to degradation of DNA was measured by Brookfield D-II+ Pro viscosimeter at 5 rpm. Results showing increased stability for DNase when stored in the presence of empty liposomes or liposomal encapsulated antiinfective are presented in FIG. 2.

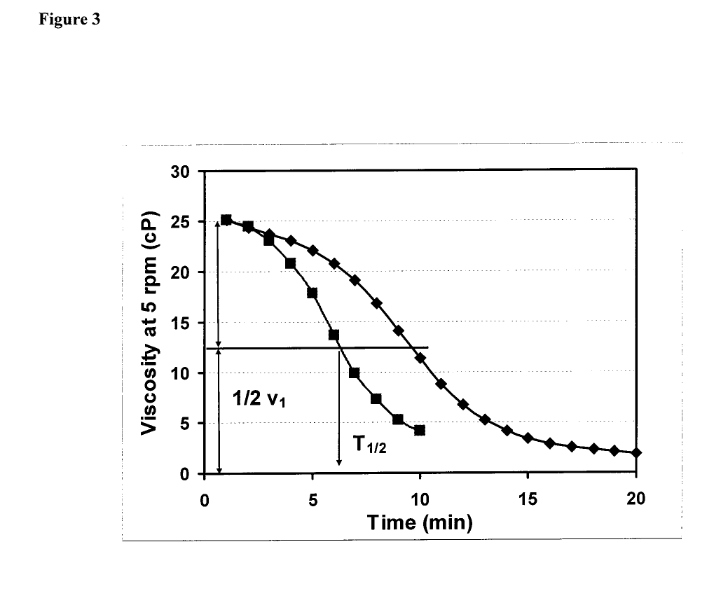

Activity was determined as a reciprocal value of a time to reduce viscosity in half, normalized to activity of standard DNase solution (A=T_{1/2}^{std}/T_{1/2}). The two curves in FIG. 3 illustrate an activity assay and how T_{1/2} is determined. The curves represent typical change in viscosity of DNA solution after DNase is added. v1 is viscosity at 1 minute, when the first measurement point is taken. T_{1/2} is the time interval after adding DNase required to reach viscosity level equal to half of v1.

### Example 4

Stability of enzymes during nebulization. Pulmozyme® was mixed with either NaCl 1.5% solution or antiinfective-encapsulated liposomes (Liposome-Amikacin, 50 mg/mL lipid concentration, 75 mg/mL amikacin concentration) suspended in NaCl 1.5%, at a volume ratio of 2:3. Liposomes were made as described previously, and had lipid composition DPPC/Cholesterol 2:1 wt/wt.

5 mL of each prepared above sample was nebulized for 10 minutes using PARI LC-Star nebulizer and DeVILBISS 8650D aerosol compressor at a pressure 30 PSI. Nebulized aerosol was collected using midjet impinger and analyzed for DNase enzymatic activity as described previously.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1-212. (canceled)

213. A composition comprising an enzyme and a liposome.

214. The composition of claim 213, wherein the enzyme is encapsulated in the liposome.

215. The composition of claim 213, wherein the enzyme is free.

216. The composition of claim 214, further comprising a free enzyme.

217. The composition of claim 1, further comprising an antiinfective, wherein the antiinfective is encapsulated in the liposome, is free, or both.

218. The composition of claim 217, wherein the enzyme is free and the antiinfective is free.

219. The composition of claim 217, wherein the enzyme is free and the antiinfective is encapsulated in the liposome.

220. The composition of claim 217, wherein the enzyme is encapsulated in the liposome and the antiinfective is encapsulated in a second liposome.

221. The composition of claim 217, wherein the enzyme is encapsulated in the liposome and the antiinfective is free.

222. The composition of claim 221, further comprising a free enzyme.

223. The composition of claim 217, wherein the enzyme is free, a portion of the antiinfective is free and another portion of the antiinfective is encapsulated in the liposome.

224. The composition of claim 217, further comprising empty liposomes.

225. A composition according to claim 213, wherein the liposome comprises lipids selected from the group consisting of phospholipids, tocopherols, sterols, glycoproteins, and mixtures thereof.

226. The composition of claim 213, wherein the liposome comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleylphosphatidylcholine (DOPC), dimyristoylphosphatidylcholine (DMPC), dimyristoylphosphatidylglycerol (DMPG), dioleylphosphatidylglycerol (DOPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and mixtures thereof.

227. The composition of claim 213, wherein the liposome comprises phospholipids and a sterol.

228. The composition of claim 213, wherein the liposome comprises dioleylphosphatidylcholine (DOPC), dioleylphosphatidylglycerol (DOPG), and cholesterol.

229. The composition of claim 228, wherein the ratio of DOPC:DOPG:cholesterol is 60-70:1-10:25-35 by mole percent.

230. The composition of claim 229, wherein the ratio of DOPC:DOPG:cholesterol is 65:5:30 by mole percent.

231. The composition of claim 213, wherein the enzyme is a lytic agent.

232. The composition of claim 213, wherein the enzyme is DNase.

233. The composition of claim 214, wherein the enzyme is DNase, and the liposome comprises lipids in a 20-30:1 ratio by weight with DNase.

234. The composition of claim 233, wherein the liposome comprises lipids in a 25:1 ratio by weight with DNase.

235. The composition of claim 233, wherein the liposome comprises DOPC, DOPG, and cholesterol at 65:5:30 mole percent, and wherein the weight ratio of DOPC, DOPG, and cholesterol to DNase is 25:1.

236. The composition of claim 217, wherein the antiinfective is an aminoglycoside.

237. The composition of claim 236, wherein the antiinfective is amikacin, gentamicin, or tobramycin.

238. The composition of claim 237, wherein the antiinfective is amikacin.

239. The composition of claim 224, wherein the empty liposomes comprise lipids selected from the group consisting of phospholipids, tocopherols, sterols, glycoproteins, and mixtures thereof.

240. The composition of claim 220, wherein the second liposome comprises lipids selected from the group consisting of phospholipids, tocopherols, sterols, glycoproteins, and mixtures thereof.

241. The composition of claim 240, wherein the antiinfective is amikacin and the second liposome comprises phospholipids and a sterol.

242. A pharmaceutical composition comprising the composition of claim 213 and a pharmaceutically acceptable carrier.

243. A method of treating pneumonia, bronchitis, cystic fibrosis, or emphysema in a subject comprising administering to a subject in need thereof a therapeutically effective amount of the pharmaceutical composition of claim 246.

244. The method of claim 243, wherein the pharmaceutical composition is administered by aerosolization.

245. The method of claim 243, wherein the pharmaceutical composition is administered daily.

246. The method of claim 243, wherein the subject is a primate, bovine, ovine, equine, porcine, rodent, feline, or canine.

247. The method of claim 246, wherein the subject is a human.

248. A method of stabilizing the activity of an enzyme comprising storing the enzyme in the presence of a liposome.

249. The method of claim 248, wherein the enzyme is a lytic agent.

250. The method of claim 249, wherein the enzyme is DNase.

251. The method of claim 248, wherein the liposome is an empty liposome.

252. The method of claim 248, wherein the liposome encapsulates an antiinfective.

253. The method of claim 248, wherein storing is conducted over 1, 2, or 3 weeks.

254. A method of stabilizing the activity of an enzyme during and after nebulization comprising nebulizing the enzyme in the presence of a liposome.

255. The method of claim 254, wherein the enzyme is a lytic agent.

256. The method of claim 255, wherein the enzyme is DNase.

257. The method of claim 254, wherein the liposome is an empty liposome.

258. The method of claim 254, wherein the liposome encapsulates an antiinfective.
